(19) United States
(12) Patent Application Publication
(10) Pub. No.: US 2023/0372232 A1
(43) Pub. Date: Nov. 23, 2023

(54) ORAL CARE COMPOSITION COMPRISING SWEET POTATO-DERIVED POTATO SYRUP OR SUPERNATANT OF POTATO SYRUP

(71) Applicants: Mitsuo KAGAYA, Tokyo (JP); KARAIMO NOJYO CO., LTD., Kyusyu-shi, Kagoshima (JP)

(72) Inventors: Osamu YAMAKAWA, Saitama (JP); Makoto YOSHIMOTO, Miyazaki (JP); Tadashi MIURA, Saitama (JP); Megumi KIDO, Kagoshima (JP); Yoshiaki HARA, Yangon (MM)

(21) Appl. No.: 18/248,254

(22) PCT Filed: Oct. 4, 2021
(86) PCT No.: PCT/JP2021/037493

§ 371 Date: Aug. 8, 2023

(30) Foreign Application Priority Data
Oct. 9, 2020 (JP) 2020-171110

(51) Int. Cl. A61K 8/9789; A61Q 11/00; A61K 36/39

(52) U.S. Cl. CPC A61K 8/9789; A61Q 11/00; A61K 36/39

(57) ABSTRACT

An oral care composition is provided. The oral care composition is configured by containing a sweet potato-derived potato syrup or a supernatant of a potato syrup.

### TECHNICAL FIELD

The present invention relates to an oral care composition, particularly specifically, relates to an oral care composition having anti-caries, anti-periodontal disease actions configured by containing a sweet potato-derived potato syrup or a supernatant of a centrifuged potato syrup (hereinafter, may simply be referred to as a supernatant).

### BACKGROUND ART

Caries (tooth decay) is a lifestyle-related disease affecting 70 percent of the world population and 90 percent of the Japanese.

Also in our country, while Ministry of Health, Labor and Welfare and Japan Dental Association have promoted the 8020 Campaign, which encourages people to “maintain 20 or more of their own teeth at the age of 80,” approximately 80 percent of the causes of tooth loss of the Japanese is still occupied by the caries and the periodontal diseases.

Furthermore, in recent years, causes of pneumonia, which is the third cause of death of the Japanese, include aspiration pneumonia of elderly people over 75 years of age, and the main causes of the aspiration pneumonia include oral bacteria, mainly periodontopathic bacteria. Therefore, in order to maintain healthy teeth even at old age and to extend healthy life expectancy, it is needless to say that the changes in eating habits and behaviors from young age are necessary, but it is also necessary to take proactive preventive measures in daily lives, such as preventing the formation of dental plaque (hereinafter, may simply be referred to as plaque or tooth plaque), which causes the caries and the periodontal diseases.

Recently, WHO (World Health Organization) has announced a new draft guideline regarding sugars intake.

It recommends that the intake of sugars be restricted to less than 5% of energy intake of a day to solve problems of obesity and diabetes, caries, and the like. According to this draft guideline, the amount of sugar that an adult of normal figure is allowed to take a day is approximately 25 g, which reaches the intake restriction only with approximately six tea spoons of sugar. Currently, sugar is used worldwide as a sweetening resource, but it is a commonly known fact that an excessive intake of sugar causes the caries. For such reasons, the inventors have developed potato syrups made mainly from sweet potatoes as a novel sweetener. Meanwhile, the inventors also have, so far, focused, in particular, on cariogenic bacteria as a main cause of the caries occurrence and numerous periodontopathic bacteria as a cause of the periodontal disease occurrence among many pathogenic germs present so as to study mechanisms of caries and periodontal disease occurrence and preventions thereof, thus having continuously made intensive effort in improving the problems described above.

A generally-well-known cariogenic bacteria as a cause of caries occurrence is Streptococcus mutans (Streptococcusmutans, hereinafter, may be referred to as Sm). Many means have been proposed so far for methods of preventing or treating the caries occurrence against the Streptococcus mutans.

The Streptococcus mutans in bacterial flora adhering on a surface of a tooth as plaque (tooth plaque), which is a typical biofilm, produces glycocalyx from sucrose, further grows therein and produces acid as a by-product, which dissolves minerals, such as calcium ions and phosphate ions out of hydroxyapatite crystal as a main component of enamel or dentin constituting a surface portion of a tooth and makes a hole on the surface of the tooth, thus, the caries occurs.

On the other hand, the periodontal diseases consequently develop into an infectious disease as a periodontal disease (alveolar pyorrhea) dissolving an alveolar bone in such a way that biofilm related bacteria described above form plaque (tooth plaque) in a periodontal pocket between a tooth and a gingiva, the plaque grows larger, specific periodontopathic bacteria typified by, for example, Porphyromonas gingivalis, (hereinafter, which may simply be referred to as Pg) grown therein deepen the periodontal pocket and cause an inflammation, and this inflammation expands over a whole periodontal tissue to be periodontitis.

Therefore, in a basic tenet of hygiene control of teeth, a main object is to create methods for suppressing the growth of this Streptococcus mutans, which is referred to as mutans streptococci described above, or environments in which this plaque (tooth plaque) is not formed on a tooth surface, that is, environments in which the plaque formation is inhibited.

In practice, there have been used plaque formation inhibiting methods and the like by, as well as fastidiously brushing teeth, mixing an agent or a composition that suppresses the growth of the Streptococcus mutans in a toothpaste or mixing a fluorine-based compound in a toothpaste so as to form a coating by the fluorine-based compound on the tooth surface.

As another method, sugar alcohols, such as xylitol as one kind of sugar alcohol, do not produce acid, thus being known to have anti-caries properties, and they are disclosed as an anti-caries agent in, for example, JP-A-9-238642 (Patent Document 1) and the like.

Green tea extract polyphenols have also been used for food products for caries prevention and periodontal disease prevention and for oral care compositions, and are disclosed in, for example, JP-A-8-81380 (Patent Document 2), JP-A-9-110687 (Patent Document 3), JP-A-11-302142 (Patent Document 4), or the like.

For the oral care compositions for periodontal disease prevention, there have been generally used products containing fungicides, such as ethanol, methyl salicylate, thymol, sodium laurate, povidone iodine, and chlorhexidine gluconate, in mouthwashes and gargling agents. For example, there is a commercially-available mouthwash as “LISTERINE” (registered trademark), which is used for caries prevention and periodontal disease prevention.

Meanwhile, for sweet potatoes, there have been disclosed a functional food, a functional ingredient, an antioxidant agent, a hepatoprotective agent, a tyrosinase inhibitor, a sugar absorption inhibitor, a neutral fat absorption inhibitor, and the like that use a sweet potato foliage extract (Patent Document 5: WO 2006/014028). For a naturally derived sugar, there is disclosed an anti-caries agent using blackstrap molasses of sugarcane (Patent Document 6: JP-A-2008-201725).

Furthermore, regarding the potato syrup, there is disclosed that the potato syrup has nutritional functionalities, such as antioxidant functions, suppression of blood sugar rise, and intestine regulating functions (Patent Document 7: JP-A-2015-000016).

Meanwhile, products and the like commonly available as, for example, “LISTERINE” (registered trademark), Isodine Gargle Solution (registered trademark), and ConCool F (registered trademark) as mouthwashes or gargling agents having caries and periodontal disease preventive effects have a problem of severe restriction in the usage as they contain ingredients harmful on the human body.

The xylitol has a problem that its production method is complicated to lead to an increased cost, and in addition, its usage is significantly limited from the aspects of taste and safety to the human body, and therefore, it is considered insufficient as a practical and effective caries prevention agent.

Furthermore, regarding the sweet potatoes, no well-established oral care composition for the human body is yet obtained in the technical field of oral care composition.

Accordingly, in the technical field of oral care composition, there is desired an oral care composition that has a simple composition structure, has caries prevention effects and plaque (tooth plaque) occurrence suppressive effects for the human body, or is able to surely prevent the occurrence of periodontal diseases, moreover, is low in cost, has no restriction in usage environments, is not harmful to the human body and has no restriction in the number of uses, and is able to be produced at a low price.
Patent Document 1: JP-A-9-238642
Patent Document 2: JP-A-8-81380
Patent Document 3: JP-A-9-110687
Patent Document 4: JP-A-11-302142
Patent Document 5: WO 2006/014028
Patent Document 6: JP-A-2008-201725
Patent Document 7: JP-A-2015-000016

### DISCLOSURE OF THE INVENTION

### Problems to be Solved by the Invention

It is an object of the present invention to provide a novel naturally derived oral care composition that suppresses occurrence and growth of caries (tooth decay) and periodontal diseases and promises prevention effects, a producing method thereof, and raw materials thereof.

It is also an object of the present invention to provide an oral care composition that uses easily available raw materials to simplify the raw material production and saves the production cost, has a simple composition structure, furthermore, has no restriction in usage environments as well as no restriction in the number of uses, and is able to be produced at a low price.

### Solutions to the Problems

As the result of intensive studies executed to solve the above-described problems, the inventors have found that a sweet potato-derived potato syrup as it is effectively suppresses growth of Streptococcus mutans (Sm) as a cariogenic bacterium and inhibits formation of tooth surface plaque (tooth plaque), and also effectively suppresses growth of Porphyromonas gingivalis (Pg) as a periodontopathic bacteria existing in plaque formed in a periodontal pocket. The inventors have found that a supernatant (hereinafter, may simply referred to as a supernatant of potato syrup) obtained by centrifuging a potato syrup solution and a potato syrup with malt produced using malt as a diastatic enzyme instead of β-amylase derived from sweet potatoes also have a similar effects, thereby completing the present invention.

That is, the present invention is an oral care composition that contains a sweet potato-derived potato syrup or a supernatant of a potato syrup. In particular, it is an oral care composition for anti-caries and/or anti-periodontal diseases.

The present invention is also an oral care composition that further contains at least one auxiliary ingredient in addition to the sweet potato-derived potato syrup or the supernatant of the potato syrup.

It is also an oral care composition in which the auxiliary ingredient is at least one ingredient selected from the group consisting of a surfactant, a colorant, a foaming agent, an antioxidant agent, a moistening agent, a sweetener, a flavoring agent, a pH adjuster, a preservative, an antiseptic agent, a solvent, an excipient, a binder, a viscous agent, an abrasive, and an extending agent.

It is also an oral care composition in which the oral care composition has one form selected from a liquid form, a viscous liquid form, a foam form, a solid form, a jelly form, a paste form, a powder form, and a powder and granular form (granular form).

It is also an oral care composition in which the oral care composition contains 0.1 to 100 mass % of the potato syrup or the supernatant of the potato syrup.

Furthermore, the present invention is an anti-caries agent and/or an anti-periodontal disease agent containing a sweet potato-derived potato syrup or a supernatant of a potato syrup.

### Effects of the Invention

The sweet potato-derived potato syrup or the supernatant of the centrifuged potato syrup solution used in the present invention has anti-cariogenicity and has antimicrobial properties to periodontopathic bacteria, thus having caries (tooth decay) prevention effects and plaque (tooth plaque) occurrence suppressive effects or is effective as an oral care composition with which occurrence of periodontal diseases is preventable.

The potato syrup or its supernatant of the present invention is refined from natural sweet potatoes and is extremely gentle on the human body, and its usage, using time, number of uses, amount of use, and the like are also not practically restricted, and therefore, it is widely usable conveniently at anytime and anywhere with safety and ease.

Unlike specific chemical compositions and compounds expected to be effective against periodontopathic bacteria, the sweet potato-derived potato syrup or its supernatant used in the present invention can, without undergoing a process of being generated or extracted, a process of mixing other ingredients including them, and the like under complicated treatment processes or complicated process conditions, maintain extremely high operation efficiency and is also possible to set a production cost at a substantially low price.

### BRIEF DESCRIPTION OF THE DRAWINGS

FIG. 1 is a drawing illustrating a comparative test result of anti-caries actions of potato syrups used in an oral care composition according to the present invention.

FIG. 2 is a drawing illustrating a comparative test result of suppressive actions on periodontopathic bacteria growth of potato syrups used in the oral care composition according to the present invention.

FIG. 3 is a drawing illustrating a comparative test result of plaque (tooth plaque) formation inhibitory actions of the potato syrups used in the oral care composition according to the present invention.

FIG. 4 is a drawing illustrating an effect on the plaque (tooth plaque) formation inhibitory actions depending on differences in concentrations of the potato syrups used in the oral care composition according to the present invention.

FIG. 5 is a drawing illustrating survival rates of periodontopathic bacteria Pg depending on the differences in concentrations of the potato syrup used in the oral care composition according to the present invention.

FIG. 6 is a drawing illustrating the suppressive actions on the growth of the periodontopathic bacteria Pg depending on differences in kinds of potato syrups used in the oral care composition according to the present invention.

FIG. 7 is a drawing illustrating a test result comparing plaque (tooth plaque) formation inhibitory actions between the potato syrup used in the oral care composition according to the present invention and a potato syrup in which malt is mixed as a diastatic enzyme.

FIG. 8 is a drawing illustrating the suppressive actions on the growth of the periodontopathic bacteria Pg by a supernatant of the potato syrup used in the oral care composition according to the present invention.

### DESCRIPTION OF PREFERRED EMBODIMENTS

The present invention is an oral care composition containing a sweet potato-derived potato syrup or a supernatant of a potato syrup. In particular, it relates to an oral care composition for anti-caries and/or anti-periodontal diseases.

It also relates to a method for producing the oral care composition. The following describes a configuration for carrying out the present invention. The present invention is not limited to the following description.

The present invention includes the following aspects [1] to [6].
[1] A first aspect of the present invention is an oral care composition containing a sweet potato-derived potato syrup or a supernatant of a potato syrup.
[1-1] An aspect 1-1 of the present invention is an oral care composition for anti-caries and/or anti-periodontal diseases containing a sweet potato-derived potato syrup or a supernatant of a potato syrup.
[2] A second aspect of the present invention is the oral care composition according to the aspect [1] further containing at least one auxiliary ingredient in addition to the sweet potato-derived potato syrup or the supernatant of the potato syrup.
[3] A third aspect of the present invention is the oral care composition according to the aspect [2] in which the auxiliary ingredient is at least one auxiliary ingredient selected from the group consisting of a surfactant, a colorant, a foaming agent, an antioxidant agent, a moistening agent, a sweetener, a flavoring agent, a pH adjuster, a preservative, an antiseptic agent, a solvent, an excipient, a binder, a viscous agent, an abrasive, and an extending agent.
[4] A fourth aspect of the present invention is the oral care composition according to any one aspect of the aspects [1] to [3] having one form selected from the group consisting of a liquid form, a viscous liquid form, a foam form, a solid form, a jelly form, a paste form, a powder form, and a powder and granular form (granular form).
[5] A fifth aspect of the present invention is the oral care composition according to any one aspect of the [1] to [4] containing 0.1 to 100 mass % of the potato syrup or the supernatant of the potato syrup.
[6] A sixth aspect of the present invention is an anti-caries agent and/or an anti-periodontal disease agent containing a sweet potato-derived potato syrup or a supernatant of a potato syrup.

The following describes the present invention in detail.

A sweet potato as a raw material of the potato syrup used in the present invention may be from any product district and may be any breed variety, but it is preferred that the breed varieties, Anno, Koganesengan, or Eimurasaki be used. For example, it is also possible to use conventionally-known domestic and foreign varieties, such as Koukei 14 Shiroyutaka, Beniharuka, Murasakimasari, Ayakomachi, Suiou, J-Red, and Beauregard.

Other tuberous roots (potatoes) containing functional components, such as Yacón and Caiapo Potato, may also be used for the oral care composition of the present invention.

As for the sweet potato used in the present invention, a main body part, that is, a tuberous part of the sweet potato is mainly used, and since their shape, size, color, breed variety, and the like are not limited, it is possible to use a low grade potato (scrap product), which is low in commercial value.

As for the sweet potato used in the present invention, while it is preferred that the main body part, that is, the tuberous part of the sweet potato be mainly used, it is also possible to use any part of a stem part, a petiole part, or a leaf part of the sweet potato alone or by being mixed in the main body part in the state where they are mixed together.

While the potato syrup used in the present invention is obtained by processing and refining the sweet potato in an appropriate method, the processing method for producing the potato syrup is not specified, and it is produced and processed using known means, such as a heating process, a pressurization process, a drying process, a steaming process, a stewing process, a shredding process, a compressing process, a kneading process, a squeezing process, and a scraping process, so as to be used for the oral care composition of the present invention.

While in the present invention, it is preferred that the above-described sweet potato as it is be used as a raw material, those that have been undergone a heating process after the harvest and frozen for storage or those that have been dried and powdered in a conventionally-known method, such as hot-air drying and heat drying under reduced pressure may be used as a raw material.

While the conditions of producing or refining the potato syrup used for the oral care composition of the present invention is based on the method disclosed in Japanese Patent No. 5981885 possessed by one of the applicants of this application, for example, the potato syrup may be produced by adding diastatic enzymes other than those derived from the sweet potatoes and performing a processing treatment in a conventionally-known producing method.

In producing the potato syrup from the sweet potatoes, what is used as the diastatic enzyme is an important technical element. In the method of this patent, using a component derived from sweet potatoes as the diastatic enzyme ensures producing a high-quality potato syrup. On the other hand, for example, it is also possible to use diastatic enzymes other than those derived from the sweet potatoes, such as those derived from a wheat or a soybean, as the diastatic enzyme.

The kind of the supernatant of the sweet potato-derived potato syrup used in the present invention is not specified as long as it is a supernatant of the potato syrup produced from the above-described breed varieties and in the above-described methods.

The extraction method of the supernatant of the potato syrup used in the present invention is preferably, under extraction conditions of the supernatant with twice the amount of the weight of the potato syrup, adding the same amount of water as the weight of the potato syrup and centrifuging the mixture at 15000 rotation per minute for approximately three to five minutes. It is also possible to extract the supernatant with water in 2 to 10 times the amount of the potato syrup.

This supernatant of the potato syrup is, besides being used as it is, also possible to be a granular, powdery oral care composition derived from sweet potatoes by further drying it in a method of, for example, freeze-drying and vacuum drying, and granulating and powdering.

The sweet potato-derived potato syrup used in the present invention has a feature as a natural sweetener with a Brix value Z of 65% to 85%.

This oral care composition of the present invention may contain one kind alone or two or more kinds of optional ingredients that are allowed to be added in the oral care composition within a range in which the advantageous effects of invention are not impaired. Such candidates are preferred to have guaranteed safety at least for the human body.

For example, the oral care composition of the present invention may further contain one kind alone or two or more kinds of: vitamin E, such as tocopherol nicotinate; enzymes, such as protease, mutanase, lysozyme, and lytic enzyme; and medicinal ingredients, such as glycyrrhetinic acid, glycyrrhizic acid, sodium fluoride, chlorophyll, and furthermore, lactoferrin.

For example, the oral care composition of the present invention may further contain one kind alone or two or more kinds of: plant extracts of, for example, lemongrass, palmarosa, and hinoki cypress; crude drugs, such as rhubarb, licorice, angelica acutiloba, and aloe; vitamins, such as vitamins B, C, and E; and known naturally derived medicinal ingredients for oral care, such as lactic acid bacteria, lactoferrin, omega-3 fatty acid, and polyphenol.

While the content of the above-described auxiliary ingredient is determined by the usage, the kind, the number of uses, and the like, it can usually be added at a proportion of 0.001 to 1 mass %, preferably 0.001 to 0.1 mass % with respect to the whole composition amount.

The auxiliary ingredient used for the present invention is at least one ingredient selected from the group consisting of a surfactant, a colorant, a foaming agent, an antioxidant agent, a moistening agent, a sweetener, a flavoring agent, a pH adjuster, a preservative, an antiseptic agent, a solvent, an excipient, a binder, a viscous agent, an abrasive, and an extending agent.

The surfactant used in the present invention is only necessary to have no effect on the human body and, for example, examples of a non-ionic surfactant include, sugar fatty acid esters, such as sucrose fatty acid ester and maltose fatty acid ester, and fatty acid monoglyceride. The blending amount is usually 0.1 to 5 mass % with respect to the whole composition amount.

Examples of the colorant used for the present invention include, for example, certified colors, such as Food Blue No. 1, Food Yellow No. 4, Food Red No. 202, and Food Green No. 3; mineral pigments, such as ultramarine, enhanced ultramarine, Prussian blue; and titanium oxide.

For the foaming agent used for the present invention, for example, a combination of at least one kind of carbonate, hydrogen carbonate, and double salt of hydrogen carbonate and carbonate (hereinafter, may be denoted as a carbonic acid compound) and acid is exemplified. While the carbonate is not specifically limited, for example, alkali metal salt of carbonate, such as sodium carbonate and potassium carbonate, is included.

While the hydrogen carbonate is not specifically limited, for example, alkali metal salt of hydrogen carbonate, such as sodium hydrogen carbonate and potassium hydrogen carbonate, is included. While the double salt of the carbonate and the hydrogen carbonate is not specifically limited, for example, sodium sesquicarbonate is included. These carbonic acid compounds may be one kind and used alone or may be used by combining two or more kinds. While the acid is not specifically limited, for example, organic acids, such as citric acid, tartaric acid, fumaric acid, malic acid, maleic acid, gluconic acid, and succinic acid; and inorganic acids, such as phosphoric acid and sulfamic acid, are included. These acids may be one kind and used alone or may be used by combining two or more kinds.

Examples of the antioxidant agent used for the present invention include, for example, a rosemary extract, a stevia extract, a sunflower seed extract, propyl gallate, dibutylhydroxytoluene, butylhydroxyanisole, L-cysteine hydrochloride, phytic acid, hydroquinone and its glycoside, nordihydroguaiaretic acid, gum guaiac, polyphenol, tocopherol acetate, a pine extract, ascorbic acid, and the like.

Examples of the moistening agent used for the present invention include, for example, sorbitol, ethylene glycol, propylene glycol, glycerin, 1,3-butylene glycol, polypropylene glycol, xylitol, maltitol, lactitol, polyoxyethylene glycol, and the like, and one kind thereof alone or two or more kinds thereof in combination can be blended.

Examples of the sweetener used for the present invention include, for example, saccharin sodium, acesulfame potassium, stevioside, neohesperidin dihydrochalcone, perillartine, thaumatin, aspartylphenylalanine methyl ester, and p-methoxycinnamic aldehyde. The blending amount is usually 0.01 to 1 mass % with respect to the whole composition amount.

Examples of the flavoring agent used for the present invention include, for example, menthol, carboxylic acid, anethole, eugenol, methyl salicylate, limonene, ocimene, n-decyl alcohol, citronellol, α-terpineol, methyl acetate, citronellyl acetate, methyl eugenol, cineole, linalool, ethyl linalool, thymol, a spearmint oil, a peppermint oil, a lemon oil, an orange oil, a sage oil, a rosemary oil, a cinnamon oil, a perilla oil, a wintergreen oil, a clove oil, a eucalyptus oil, a pimento oil, d-camphor, d-borneol, a fennel oil, a cinnamon oil, cinnamaldehyde, a mint oil, and vanillin. The blending amount of one kind alone or a combination of two or more kinds is usually, for example, 0.001 to 5 mass % with respect to the whole composition amount.

Examples of the pH adjuster used for the present invention include, for example, citric acid, phosphoric acid, malic acid, pyrophosphoric acid, lactic acid, tartaric acid, glycerophosphoric acid, acetic acid, nitric acid, or their chemically possible salts, sodium hydroxide, and the like. They can be alone or blended by combining two or more kinds such that the composition has a pH within a range of 4 to 8, preferably 5 to 7. The blending amount of the pH adjuster may, for example, be 0.01 to 2 mass %.

Examples of the preservative used for the present invention include, for example, parabens (e.g., methylparaben, ethylparaben, propylparaben, and butylparaben), benzoic acid, sodium benzoate, dehydroacetic acid, sodium dehydroacetate, isobutyl parahydroxybenzoate, isopropyl parahydroxybenzoate, butyl parahydroxybenzoate, ethyl parahydroxybenzoate, propyl parahydroxybenzoate, benzyl parahydroxybenzoate, methyl parahydroxybenzoate, phenoxyethanol, alkyldiaminoethylglycine hydrochloride, benzalkonium chloride, and benzethonium chloride.

Examples of the preservative used for the present invention include, for example, parabens, such as methylparaben, ethylparaben, propylparaben, and butylparaben, sodium benzoate, phenoxyethanol, and alkyldiaminoethylglycine hydrochloride.

Examples of the solvent used for the present invention include, for example, ethanol, propylene glycol, butylene glycol, and hexylene glycol.

Examples of the excipient used for the present invention include, for example, reduced palatinose, palatinose, sorbitol, reduced maltose starch syrup, erythritol, trehalose, mannitol, xylitol, crystalline cellulose, gum base, gelatin, and gum Arabic, and can be used alone or by combining two or more kinds as necessary. Although their blending amounts are different depending on dosage forms, the blending amount is usually 5 to 95 mass % with respect to the whole composition amount.

Examples of the binder used for the present invention include, for example, a cellulose derivative, sodium alginate, carrageenan, and xanthan gum, and can be used alone or by combining two or more kinds as necessary. The blending amount is usually 0.5 to 5 mass % with respect to the whole composition amount.

Examples of the thickening agent used for the present invention include, for example, cellulose derivatives, such as carboxymethylcellulose sodium, sodium carboxymethyl hydroxycellulose, hydroxyethyl cellulose, hydroxypropyl cellulose, cationized hydroxyethyl cellulose, and crystalline cellulose; alkali metal alginates, such as sodium alginate; gums, such as propylene glycol alginate, xanthan gum, gum tragacanth, karaya gum, gum arabic, and carrageenan; synthetic thickening agents, such as polyvinyl alcohol, sodium polyacrylate, carboxyvinyl polymer, and polyvinylpyrrolidone; inorganic thickening agents, such as silica gel, aluminum silica gel, VEEGUM, and laponite.

Examples of the abrasive used for the present invention include, for example, calcium hydrogen phosphate, calcium pyrophosphate, calcium carbonate, silica, alumina, aluminosilicate, aluminum hydroxide, zeolite, and kaolin. They can be used alone or by combining two or more kinds as necessary. The blending amount is usually 1 to 40 weight % with respect to the whole composition amount.

When the sweet potato-derived potato syrup or the supernatant and the auxiliary ingredient are mixed and used in the oral care composition of the present invention, the mixture ratio of both the ingredients is not specifically limited, but it is preferred that, for example, the auxiliary ingredient be 0.001 to 1.5 mass % of the sweet potato-derived potato syrup.

It is preferred that, after the potato syrup and the auxiliary ingredient used for the present invention are individually prepared to have respective necessary amounts, both the ingredients undergo stirring and mixing process using appropriate mixing means and both the ingredients be processed to be uniformly mixed with one another.

It is preferred that the process be performed by appropriately selecting as necessary a device for the mixture process or mixing conditions in the mixture process, for example, temperature conditions, stirring conditions, and pressurizing conditions during the mixing procedure and the mixture process, the mixing process time, and the like.

The oral care composition of the present invention has one form selected from the group consisting of a liquid form, a viscous liquid form, a foam form, a solid form, a jelly form, a paste form, a powder form, and a powder and granular form (granular form).

While it is not specifically limited, as more specific commercialized form functioning as the oral care composition of the present invention, in the case of the potato syrup, for example, preferred specific examples include forms of a food product in, for example, a syrup form, or a toothpaste, a gargling agent, a mouthwash, or a candy, a gum, a drinking jelly, a beverage, a supplement, a spray, a chocolate, or an ice cream.

While it is not specifically limited, as more specific commercialized form functioning as the oral care composition of the present invention, in the case of the supernatant of the potato syrup, for example, preferred specific examples include forms of a spray mouthwash, a gargling agent, and a beverage of spray types, or a supplement of a granule and powder type.

When the powder state or granule state are employed as the final usage form of the oral care composition of the present invention, a conventionally-known method, such as freeze-drying and spray drying, is usable. While it is not specifically limited, in one example, the oral care composition of the present invention is frozen at −20° C. to −30° C. for a few hours, then, the frozen sample is put in a vacuum unit to be vacuumed at −50° C. for 24 hours, and then, only the water content is vaporized in a freeze-drying unit. Then, putting it back in a normal temperature and atmospheric pressure ensures obtaining a product in a dry powder state or granule state.

Adding and mixing the sweet potato-derived potato syrup or the supernatant of the potato syrup used for the present invention in an existing food product as a natural sweetener or an additive allows the food product to have caries and periodontal disease preventive properties. For example, examples of such a food product include beverages, seasonings, alcoholic beverages, furthermore, liquid form, syrup form, powder form, granular form, pill form, tablet form, soft capsule form, hard capsule form, jelly form, or paste form nutritional supplementary foods, foods for specified health use, functional foods, health foods, therapeutic foods for nursing or dysphagia, and the like, as well as various kinds of general processed products, such as confectioneries, noodles, processed meat and fish meat products, dairy products, and breads.

Targets of the oral care composition of the present invention include mammals (e.g., dogs) including humans, and in particular, humans are preferred.

Next, the method for producing the oral care composition functioning as the oral care composition according to the present invention is not particularly specified, but one specific example is, for example, a method for producing an oral care composition including a first step of producing a sweet potato-derived potato syrup from sweet potatoes, a second step of selecting one kind or two or more kinds of auxiliary ingredients to be mixed in the sweet potato-derived potato syrup, and a third step of performing a process of mixing the potato syrup obtained in the first step and the auxiliary ingredient selected in the second step with appropriate means.

In the method for producing the oral care composition of the present invention, each of conditions, such as mixture ratios of respective constituent materials; the mixing operation method; and the heating, the pressure, the rotational speed, the processing time, and the features and the mixing amount of a third ingredient to be added during the mixing in the third step is not specifically limited, and, for the component composition and the shape of the final product of the oral care composition, devices or processing treatment conditions are appropriately selected according to the properties.

Furthermore, a method for producing the oral care composition as another aspect according to the present invention includes, subsequent to the above-described third step, one or a plurality of additional steps, such as various kinds of forming process steps, a granulation or granule formation step, furthermore, a packing step, and a housing step into an appropriate container, are added according to the conditions, such as the shape or the property in the final product of the oral care composition, for example, if the final product of the oral care composition is in a liquid form or a solid form, or in a paste form or a granular form.

Examples of the oral care composition of the present invention include, for example, a gum, a candy, a jelly, a beverage, a supplement, or a food product in, for example, a syrup form; a toothpaste, a gargling agent, or a mouthwash; or a spray liquid.

What it means by “the anti-caries agent and/or the anti-periodontal disease agent containing the sweet potato-derived potato syrup or the supernatant of the potato syrup” in the present invention is the use of the sweet potato-derived potato syrup or the supernatant of the potato syrup for anti-caries and/or anti-periodontal diseases and it encompasses the oral care composition.

### EXAMPLES

While the following shows Examples and specifically describes the present invention in detail, the present invention is not limited to those.

### (Example 1) Production of Sweet Potato-Derived Potato Syrup

Based on the method disclosed in Japanese Patent No. 5981885 possessed by one of the applicants of this application, 160 Kg each of three varieties of sweet potatoes, Anno Potato, Koganesengan, and Eimurasaki, were used and were steamed and simmered for approximately one hour, and then, 75 L of water was added to make a slurry. Afterwards, this liquid was added with β-amylase obtained from Beniharuka and heated at 65° C. for 30 minutes. The obtained dissolution liquid was pressurized to extract the juice, thereby obtaining a raw material for each potato syrup. Then, the raw material was further heated to be concentrated for approximately two hours. Thus, from the Anno Potato (Example 1-A), the Koganesengan (Example 1-B), and the Eimurasaki (Example 1-C), 50 Kg, 55 Kg, and 50 Kg of potato syrups, respectively, were obtained.

The potato syrup of Example 1-A is sold as trade name: “Anno,” the potato syrup of Example 1-B is sold as trade name: “Satsuma,” and the potato syrup of Example 1-C is sold as trade name: “Murasaki” by Karaimo Farm Co., Ltd., another applicant of this application.

In this application, when denoted as “Example 1-A,” “Example 1-B,” and “Example 1-C,” they may indicate the potato syrups produced from the respective breed varieties.

### (Example 2) Production of Supernatant of Sweet Potato-Derived Potato Syrup

3 g of the potato syrup (Eimurasaki, Example 1-C) obtained in Example 1 was diluted into 50% with an equal volume of water. The dilution was centrifuged in a test tube at 15000 rotations per minute for five minutes using a MX-305 centrifugal separator manufactured by TOMY SEIKO Co, Ltd., and then, the precipitates were removed, thereby obtaining 5.4 g of clear supernatant.

### (Example 3) Production of Potato Syrup with Malt

160 Kg of Koganesengan was used and was steamed and simmered for approximately one hour in a high temperature steam atomization state of approximately 100° C., and then, 75 L of water was added to make a slurry. Afterwards, this liquid was added with 1.6 Kg of malt (manufactured by SUNTORY MALTING LIMITED; malt for starch syrup 4/10 ground) as a diastatic enzyme, and heated at 60° C. for 30 minutes. The obtained dissolution liquid was pressurized to extract the juice, thereby obtaining a raw material for a potato syrup. Then, the raw material was further heated to be concentrated for approximately two hours, thereby obtaining 60 Kg of a potato syrup (Example 3) in which the malt was used as the diastatic enzyme.

### (Test Example 1) Anti-Caries Actions of Sweet Potato-Derived Potato Syrup

The potato syrups of Example 1-A and Example 1-C obtained in Examples described above were used, and an additive-free sample that does not contain the potato syrup was produced as a comparison target. Then, the anti-caries actions were examined.

10 mL of 1% concentration sucrose liquid was added to a brain heart infusion (hereinafter abbreviated as BHI) medium and 120000 Streptococcus mutans (Sm) were added to this, thereby producing a “basic sample S.”

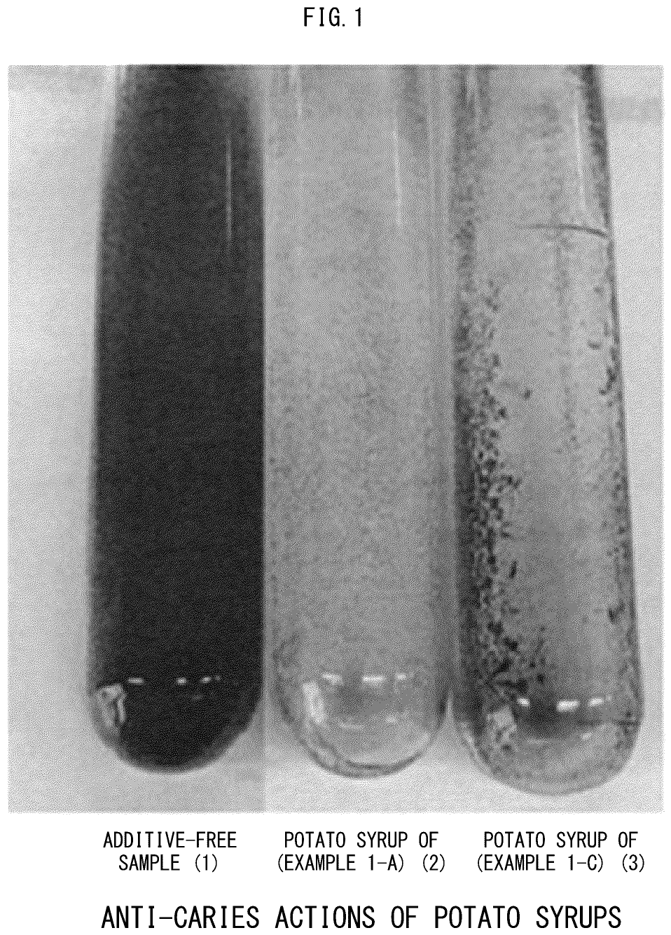

There were respectively produced a sample (additive-free sample) (1) obtained by adding nothing in this basic sample S, a sample (2) obtained by adding 1.0 g of the potato syrup of Example 1-A in the basic sample S, and a sample (3) obtained by adding 1.0 g of the potato syrup in Example 1-C in the basic sample S. After the respective samples were subject to a culture process at 37° C. for 48 hours, the culture fluids were discarded. Then, 10 mL of distilled water was added thereto, and a stirring process was performed. After the stirred liquid was discarded, crystal violet staining was performed, and thus, whether plaque (tooth plaque) was formed in each of the samples or not was examined. The results are shown in FIG. 1.

As the result of the comparative experiment of the anti-caries actions, in the sample (1) with no additive, a large amount of white turbid object occurred and was stained in dark blue, indicating the occurrence of plaque (tooth plaque). In contrast, in the samples (2) and (3) in which the potato syrups according to the present invention were used, little or no white turbidity were observed, indicating no occurrence of plaque (tooth plaque). Accordingly, it should be understood that the oral care composition using the potato syrup according to the present invention has anti-caries actions that inhibit the formation of plaque (tooth plaque).

### (Test Example 2) Suppressive Actions on Growth of Periodontopathic Bacteria by Sweet Potato-Derived Potato Syrup

There were prepared the potato syrups of Example 1-A, Example 1-B, and Example 1-C obtained in Examples described above and the “basic sample P” by, to 1 mL of liquid medium, obtained by adding hemin and menadione, which are necessary for growth of periodontopathic bacterium Porphyromonas gingivalis (Pg), in a TBS medium, inoculating 80 μL of Pg that has been cultured in advance in the same medium.

There were individually adjusted and produced a medium (a sample 4) produced by adding 10% of the potato syrup of Example 1-A to the basic sample P, a medium (a sample 5) produced by adding 10% of the potato syrup of Example 1-B to the basic sample P, a medium (a sample 6) produced by adding 10% of the potato syrup of Example 1-C to the basic sample P, and a medium without adding anything to the basic sample P, that is, a normal medium (a sample 7).

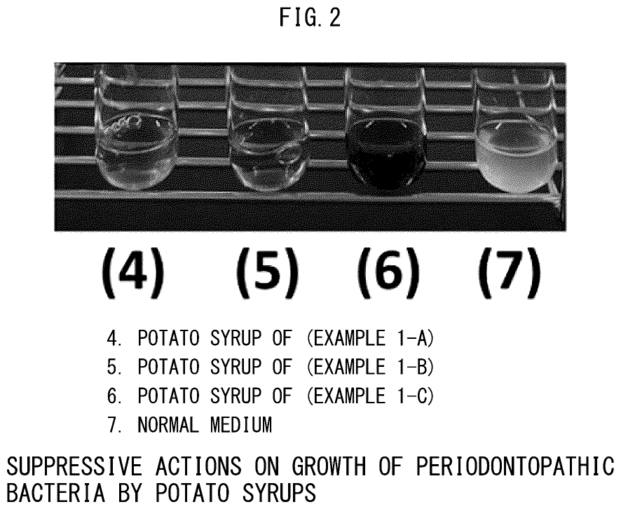

Afterwards, the respective samples were subject to an anaerobic culture process at 37° C. for two days, and then, overview observation photographs were taken for the respective samples. The results are shown in FIG. 2.

As the result, the liquids of the media in the sample (4) to sample (6) were clear, and no growth or propagation of the periodontopathic bacteria Pg was visually perceived in any of those samples. On the other hand, in the comparative normal medium (the sample 7), a white turbid cloud was present, and thus, the growth and propagation of the periodontopathic bacteria Pg was confirmed.

From this result, the oral care composition using the sweet potato-derived potato syrup according to the present invention is expected to suppress the growth of the periodontopathic bacteria and have the periodontal disease prevention effects.

### (Test Example 3) Inhibitory Actions on Tooth Plaque Formation by Sweet Potato-Derived Potato Syrup

A basic sample (a sample 8) was produced by impregnating 1% concentration sucrose (white sugar) in a BHI medium of twice the normal concentration and adding 0.1 mL of Streptococcus mutans liquid (approximately 1000 bacterial bodies) thereto.

Next, another sample (a sample 9) in which the sucrose (white sugar) was not at all impregnated in the basic sample (the sample 8) was produced. There were also individually produced a sample (10) in which 10% of the potato syrup of Example 1-A was added instead of the 1% concentration sucrose (white sugar) to the basic sample (the sample 8), a sample (11) in which 10% of the potato syrup of Example 1-B was added instead of the 1% concentration sucrose (white sugar) to the basic sample (the sample 8), and a sample (12) in which 10% of the potato syrup of Example 1-C was added instead of the 1% concentration sucrose (white sugar) to the basic sample (the sample 8). The respective samples were subject to a culture process at 37° C. for 48 hours.

The supernatant liquids of the culture fluids were discarded, and then, the respective samples were washed twice with 0.2 mL of distilled water. Afterwards, 0.2 mL each of 0.1% crystal violet was added to each of the samples, a stir staining operation was performed for 20 minutes, and then, the staining fluids were discarded. The residues were washed twice with 0.2 mL of distilled water. Then, the pigment was eluted with 0.2 mL of 99.5% ethanol, and the absorbance (570 nm) was measured.

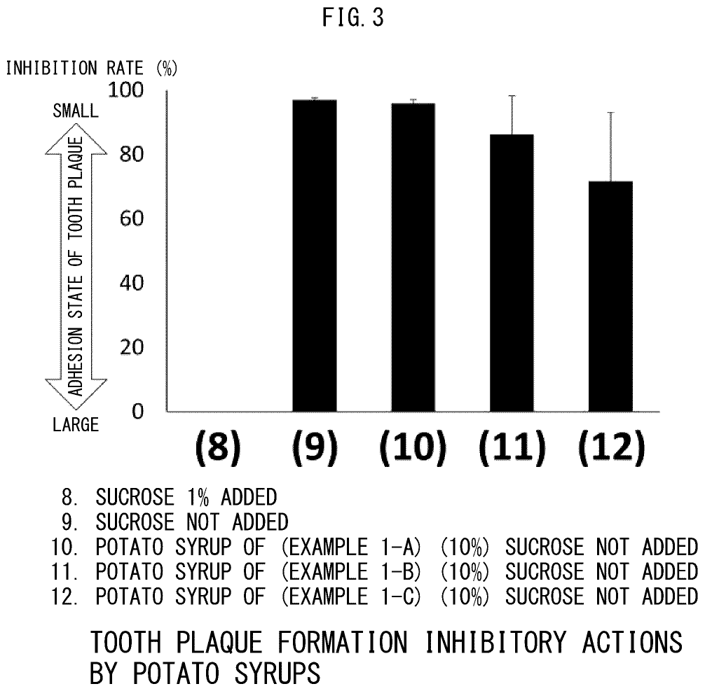

Based on the results, the plaque (tooth plaque) formation inhibition rates (%) were calculated. The results are shown in FIG. 3.

plaque formation inhibition rate (%)=[A570(no sucrose added)−A570 (sample added)]/A570(no sucrose added)×100

A570: absorbance of crystal violet eluted with ethanol at 570 nm.

The result showed that, in the case where the sucrose was present (the sample 8), this sucrose fed the cariogenic bacteria, and the plaque (tooth plaque) rapidly grew, whereas in the case where the sucrose was not present (the sample 9), little or no plaque (tooth plaque) was formed. It was also shown that, in the case where the potato syrups were used (the samples 10 to 12), little or no plaque (tooth plaque) was formed similarly to (the sample 9). The potato syrup was shown to exert the plaque (tooth plaque) formation inhibitory effects.

### (Test Example 4) Effect on Tooth Plaque Formation Inhibitory Actions Related to Concentration of Sweet Potato-Derived Potato Syrup

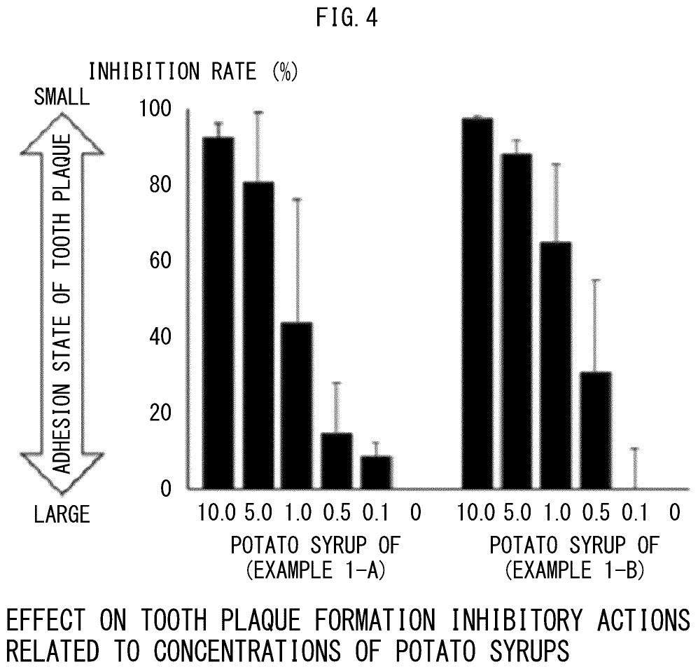

The potato syrups of Example 1-A and Example 1-B used in Test Example 3 were examined in changes (effects) in the plaque (tooth plaque) formation inhibition rate depending on the concentrations of the potato syrups at the time of the respective uses. The results are shown in FIG. 4.

As the result, all of the potato syrups were able to be confirmed that, relating to the use of potato syrup at usage concentrations between 0.1% and 10%, the plaque (tooth plaque) formation inhibitory actions were high when the usage concentrations were high, and thus, the inhibitory actions were concentration dependent.

### (Test Example 5-1) Survival Rate (1) of Periodontopathic Bacteria Pg Depending on Concentration of Potato Syrup

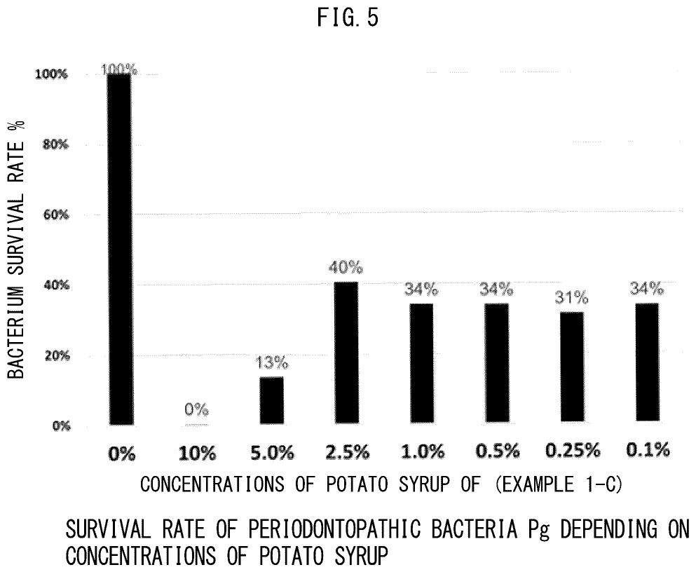

The potato syrup of Example 1-C was analyzed and examined to see how the survival rate of the periodontopathic bacteria Pg changes depending on the concentration of the potato syrup at the time of its use. The result is shown in FIG. 5.

As the result, it was shown that, when this potato syrup was not used, the periodontopathic bacteria Pg were not at all killed, and most of them survived and propagated, on the other hand, relating to the use of the potato syrup at usage concentrations between 0.1% and 10%, the survival rate of the periodontopathic bacteria certainly decreased.

In particular, it was shown that the periodontopathic bacteria Pg were certainly killed when the usage of the potato syrup was 10%, and under the condition of the potato syrup usage of 0.10% to 5%, it was shown that the survival rate of the periodontopathic bacteria Pg was able to be reduced to 40% or less.

### (Test Example 5-2) Suppressive Actions (2) on Growth of Periodontopathic Bacteria Pg Depending on Kind of Potato Syrup

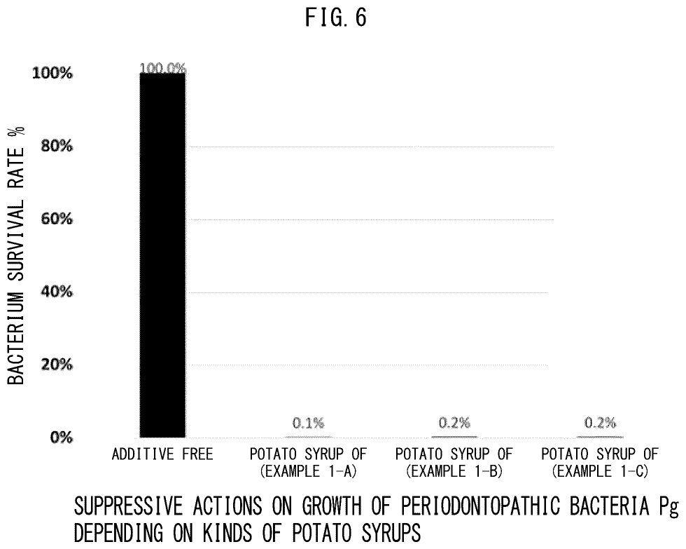

The concentrations of the potato syrup of Example 1-A, the potato syrup of Example 1-B, and the potato syrup of Example 1-C were set to 10%, and suppressive effects on the growth of the periodontopathic bacteria Pg were compared and examined. The results are shown in FIG. 6.

As the result, it was shown that no suppressive effect on the growth of the periodontopathic bacteria Pg was observed at all when no potato syrup was used, whereas, when 10% of the respective potato syrups were mixed, nearly 100% suppressive effects on the growth were provided in the respective cases.

### (Test Example 6) Tooth Plaque Formation Inhibitory Actions by Sweet Potato-Derived Potato Syrup with Malt

The potato syrup with malt obtained in Example 3 described above and the potato syrup of Example 1-B were used, and an additive-free sample that does not contain the potato syrup was produced, and then, anti-caries actions were examined. A basic sample was produced by impregnating 1% concentration sucrose (white sugar) in a BHI medium and adding 0.1 mL of Streptococcus mutans liquid (approximately 1000 bacterial bodies) thereto.

Next, another additive-free sample in which the sucrose (white sugar) was not at all impregnated in the basic sample was produced. There were also individually produced a sample in which 10% of the potato syrup with malt of Example 3 was added instead of the 1% concentration sucrose (white sugar) to the basic sample and a sample in which 10% of the potato syrup of Example 1-B was added instead of the 1% concentration sucrose (white sugar) to the basic sample. The respective samples were subject to a culture process at 37° C. for 48 hours.

Afterwards, the supernatant liquids of the culture fluids were discarded, and then, the respective samples were washed twice with 0.2 mL of distilled water. Afterwards, 0.2 mL each of 0.1% crystal violet were added to each of the samples, a stir staining operation was performed for 20 minutes, and then, the staining fluids were discarded. The residues were washed twice with 0.2 mL of distilled water. Then, the pigment was eluted with 0.2 mL of 99.5% ethanol and the absorbance (570 nm) was measured.

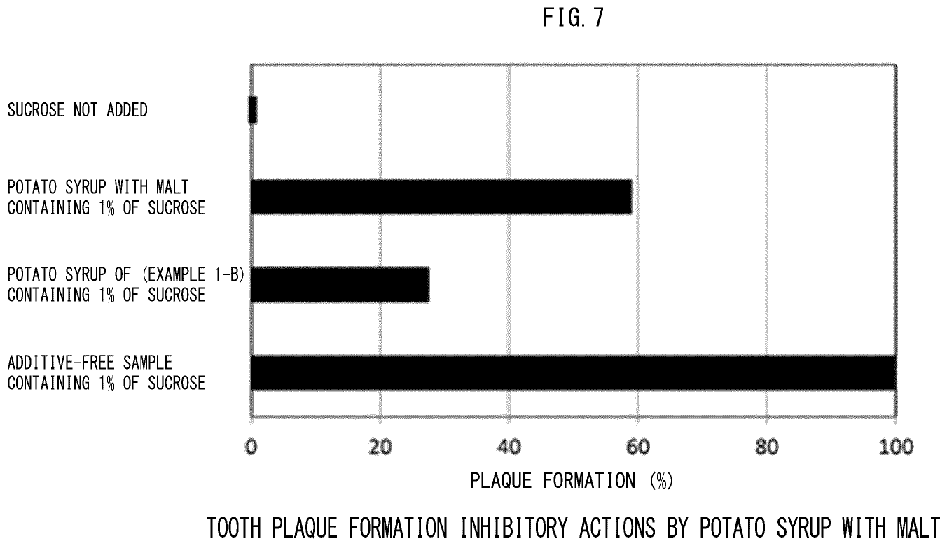

Based on the results, the plaque (tooth plaque) formation inhibition rates (%) were calculated. The results are shown in FIG. 7.

As the result, it was confirmed that, in the case where the sucrose was present, this sucrose fed the cariogenic bacteria, and the plaque (tooth plaque) rapidly grew, whereas in the case where the sucrose was not present, little or no plaque (tooth plaque) was formed. It was also shown that, also in the case where the potato syrup with malt of Example 3, little or no plaque (tooth plaque) was formed similarly to the potato syrup of Example 1-B. The potato syrup was shown to exert the plaque (tooth plaque) formation inhibitory effects.

### (Test Example 7) Suppressive Actions on Growth of Periodontopathic Bacteria Pg by Supernatant of Sweet Potato-Derived Potato Syrup

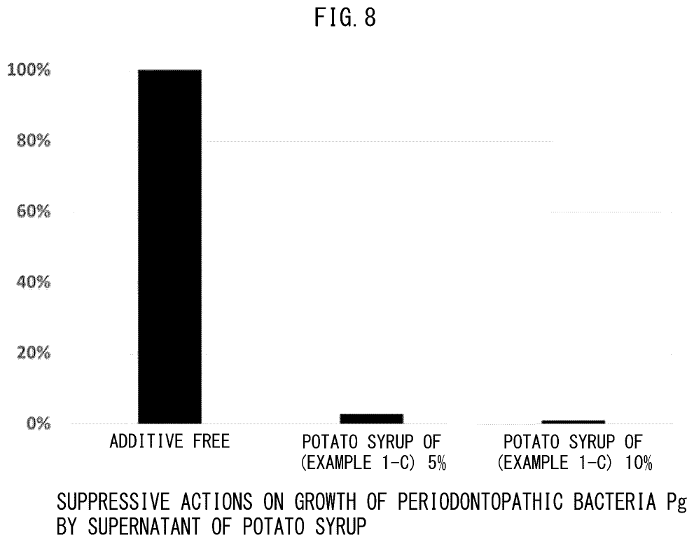

The supernatant of the potato syrup solution of Example 1-C was analyzed and examined to see how the survival rate of the periodontopathic bacteria Pg changes depending on the concentration of the supernatant at the time of its use. The result is shown in FIG. 8.

As the result, it was shown that, when the supernatant of the potato syrup solution was not used, the periodontopathic bacteria Pg were not at all killed, and most of them survived and propagated, on the other hand, relating to the use of the supernatant at usage concentrations between 5% and 10%, the survival rate of periodontopathic bacteria Pg certainly decreased.

In particular, it was shown that the periodontopathic bacteria Pg were almost certainly killed when the usage of the supernatant was 10%, and an effect similar to the growth suppression provided by the potato syrup as the main body was exhibited.

### Formulation Examples of Oral Care Composition

The following describes specific examples regarding configurations of the oral care composition of the present invention.

| | |
|---|---|
| Ingredient | Blending Amount |

(Potato Syrup Candy)

| | | |
|---|---|---|
| Potato Syrup | 70 to 80 | mass % |
| Malt Syrup | 20 to 30 | mass % |

(Potato Syrup Chocolate)

| | | |
|---|---|---|
| Potato Syrup | 10 to 30 | mass % |
| Cocoa Mass | 40 to 60 | mass % |
| Cocoa Butter | 20 to 30 | mass % |
| Milk Powder | 0 to 20 | mass % |
| Extending Agent | 5 to 10 | mass % |

0001

(Potato Syrup Gum)

| | | |
|---|---|---|
| Potato Syrup | 40 to 50 | mass % |
| Gum Base (Vegetable Resin, Such as Chicle) | 40 to 50 | mass % |
| Potato Syrup Powder | 2 to 5 | mass % |

0002

(Potato Syrup Drinking Jelly)

| | | |
|---|---|---|
| Potato Syrup | 30 to 40 | mass % |
| Water | 40 to 60 | mass % |
| Gelatinizing Agent (DJ-100) | 0.8 | mass % |
| Vitamin C | 0.4 | mass % |

(Potato Syrup Ice Cream)

| | | |
|---|---|---|
| Potato Syrup | 30 to 40 | mass % |
| Bovine Milk | 40 to 60 | mass % |
| Cream | 5 to 8 | mass % |
| Skimmed Milk | 3 to 5 | mass % |
| Stabilizer (Polysaccharide Thickener) | 0.3 to 0.5 | mass % |

0003

### INDUSTRIAL APPLICABILITY

The sweet potato-derived potato syrup or the supernatant of the potato syrup obtained by centrifuging the potato syrup solution used for the present invention have anti-cariogenicity and have antimicrobial properties to the periodontopathic bacteria, thus having the caries (tooth decay) prevention effects and the plaque (tooth plaque) occurrence suppressive effects or being effective as an oral care composition capable of preventing the occurrence of periodontal diseases.

The potato syrup or its supernatant of the present invention is refined from natural sweet potatoes and is extremely gentle on the human body, and its usage, using time, number of uses, amount of use, and the like are also not practically restricted, and therefore, it is widely usable conveniently at anytime and anywhere with safety and ease. Unlike specific chemical compositions and compounds expected to be effective against periodontopathic bacteria, the sweet potato-derived potato syrup or its supernatant used in the present invention can, without undergoing a process of being generated or extracted, a process of mixing other ingredients including them, and the like under complicated treatment processes or complicated process conditions, maintain extremely high operation efficiency and is also possible to set a production cost at a significantly low price.

The oral care composition of the present invention is an oral care composition derived from a natural product that is harmless to the human body compared with existing chemicals and artificial sweeteners, is free from side effects, and provides the caries (tooth decay) and periodontal disease preventive functions, and is usable for oral care usage simply and easily over a wide range of fields without being affected by any use restrictions.

With the oral care composition according to the present invention, a comprehensive oral care, such as prevention or therapy of caries and periodontal diseases, is possible.

Furthermore, the oral care composition according to the present invention has an excellent taste and is inexpensive in price. Therefore, it is a product for oral care with widely set usage environment, and moreover, with a possibility of a large consumption.

Meanwhile, the sweet potato used for the oral care composition according to the present invention is not restricted by product district or breed variety, shape, size, color, or the like, and most of the sweet potatoes publicly-known currently can be utilized. As the result, a large contribution is made to vitalize the domestic sweet potato industry and local industries.

In addition, at the same time, the oral care composition according to the present invention can be utilized as a health food with components, such as polyphenol, dietary fiber, and carotene, contained in the sweet potato being expected to suppress the aging of the human body caused by the reactive oxygen and the occurrence of diseases, such as cancer, diabetes, and high blood pressure, in combination.

1. An oral care composition for anti-caries and/or anti-periodontal diseases, comprising
a sweet potato-derived potato syrup or a supernatant of a potato syrup.

2. The oral care composition according to claim 1, further comprising
at least one auxiliary ingredient in addition to the sweet potato-derived potato syrup or the supernatant of the potato syrup.

3. The oral care composition according to claim 2, wherein
the auxiliary ingredient is at least one auxiliary ingredient selected from the group consisting of a surfactant, a colorant, a foaming agent, an antioxidant agent, a moistening agent, a sweetener, a flavoring agent, a pH adjuster, a preservative, an antiseptic agent, a solvent, an excipient, a binder, a viscous agent, an abrasive, and an extending agent.

4. The oral care composition according to claim 1, wherein
the oral care composition has one form selected from the group consisting of a liquid form, a viscous liquid form, a foam form, a solid form, a jelly form, a paste form, a powder form, and a powder and granular form (granular form).

5. The oral care composition according to claim 1, wherein
the oral care composition contains 0.1 to 100 mass % of the potato syrup or the supernatant of the potato syrup.

6. An anti-caries agent and/or an anti-periodontal disease agent comprising
a sweet potato-derived potato syrup or a supernatant of a potato syrup.
